(19) United States
(12) Patent Application Publication
(10) Pub. No.: US 2017/0020583 A1
(43) Pub. Date: Jan. 26, 2017

(54) SURGERY DEVICE ASSEMBLY FOR MINIMALLY INVASIVE SPINAL SURGERY

(71) Applicant: INTAI Technology Corporation, Taichung (TW)

(72) Inventors: YUNG-FANG TSAI, Taichung City (TW); DIAN-YING LIN, Hemei Township (TW); SHIH-CHANG CHUANG, Taichung City (TW); DIN-HSIANG TSENG, Changhua City (TW)

(21) Appl. No.: 15/217,159

(22) Filed: Jul. 22, 2016

(30) Foreign Application Priority Data
Jul. 24, 2015 (TW) 104123956
Jul. 24, 2015 (TW) 104123957

(51) Int. Cl. A61B 17/70; A61B 17/88

(52) U.S. Cl. CPC A61B 17/7076; A61B 17/8872; A61B 17/7032; A61B 17/708; A61B 17/7079; A61B 17/7089

(57) ABSTRACT

A surgery device assembly for minimally invasive spinal surgery is provided. The surgery device assembly includes a plurality of pedicle screws, a least one parallel bridge, a guiding device and an elongated rod. The plurality of pedicle screws are drilled into the corresponding spine respectively. Each pedicle screw is clamped by the corresponding parallel bridge in order to keep the cannulas parallel to each other. The top of the sidewalls is a U-type trough in order to adjust a direction of each pedicle screw to the same way. The proper shape of the elongated rod is bent according to the curve formed with the U-type grooves of pedicle screws. The elongated rod is seated in trough portion of each pedicle screw by puncturing the guiding device. The elongated rod is then secured by nuts in order to fix a bony structure.

### CROSS-REFERENCE TO RELATED APPLICATION

This application claims priority from Taiwan Patent Application No. 104123956 and No. 104123957, filed on Jul. 24, 2015, in the Taiwan Intellectual Property Office, the content of which is hereby incorporated by reference in their entirety for all purposes.

### BACKGROUND OF THE INVENTION

1. Field of the Invention

This application relates to a medical apparatus, and more particularly, to a pedicle screw assembly feasible for the percutaneous minimal invasive spinal fusion surgery.

2. Description of the Related Art

As far as the conventional spinal fusion surgery is concerned, the surgeon has to cut the patient's open wound with a larger area so as to obtain a better visual field to determine the position where the pedicle screw is implanted in and a direction of the recess of a U-type trough. However, an oversized wound may damage to the surrounding muscle and tissue, resulting a longer recovery time. As to the percutaneous minimal invasive spinal fusion surgery, it takes only a few hours to complete the entire surgery. Because the pedicle screw is drilled into the target spine, the U-type trough for accommodating the pedicle screw is put into the deeper tissue, so that the surgeon cannot instinctively observe the relative position therebetween other than relying on the clinical experience and tentative disposition. Consequently, the uncertainty occurred in process of the surgery is increased.

In addition, there are guiding devices provided to increase the precision and accuracy when the elongated rod is applied to penetrate through, the technical problem of the parallel arrangement of the pedicle screw is not effectively resolved. So, the current guiding devices are only designed to aim at puncturing and guiding two adjacent pedicle screws. Once there are numerous adjacent vertebral bodies needed to be fused, it has to apply the surgical navigation system. The surgical navigation system, however, is of complicated operation and costs a lot, and it is therefore hard to be applied extensively.

Besides, the convention short-tail pedicle screw has to cooperate with the other guiding devices such as a guiding sleeve in the spinal operation, but using too many medical devices may take lots of time. Consequently, when long-tail pedicle screw came out, the guiding passageway formed on two sides of the long-tail pedicle screw is applied to replace the guiding sleeve.

However, the current open type long-tail pedicle screw still has certain technical problems when being used. For example, the curve of the spine cannot be observed instinctively, resulting in an inconsistent curve connection between the elongated rod and the target spine. Moreover, whether the position among each pedicle screw aligns or not may also affect the elongated rod to penetrate. The foregoing technical problems can be resolved according to the surgeon's clinical experience, though, the medical apparatus, which is easy to be used and portable, may further simplify the surgery procedure so as to shorten the operation.

As a result, the inventor of the present disclosure has designed a surgery device assembly for minimally invasive spinal surgery to resolve the foregoing technical problems and to aim at improving the shortcomings of the current technique, so as to promote the industrial practicability.

### SUMMARY OF THE INVENTION

In view of the aforementioned technical problems, the objective of the present disclosure provides a surgery device assembly for minimally invasive spinal surgery. The surgery device assembly includes a plurality of pedicle screws, a least one parallel bridge, a puncture guiding device and an elongated rod.

In view of the aforementioned technical problems, the objective of the present disclosure provides a pedicle screw, and the surgeon can apply a U-type simulation groove disposed at the end of the pedicle screw to directly determine the relative position among each spine. In addition, the relative relationship among the U-type simulation groove of each pedicle screw is applied to compare with a curve of the elongated rod.

In view of the aforementioned technical problems, the objective of the present disclosure further provides a pedicle screw which applies at least two sets of clamping units to respectively clamp the corresponding pedicle screw, and a parallel bridge is applied to keep parallel among each pedicle screw. Besides, a small gap between the clamping unit and the long sidewalls of the pedicle screw is provided to adjust a direction of a U-type simulation groove of the pedicle screw.

In view of the aforementioned technical problems, the objective of the present disclosure provides a puncture guiding device. Before the puncture guiding device is used, each of adjusting pedicle screws is respectively adjusted on the same parallel plane, a curve of an elongated rod is adjusted to match the adjusted pedicle screw, and then a tip of the rod having a cone is disposed on the puncture guiding device to penetrate a trough portion of the pedicle screw to peel off the deep muscle tissue so as to form an trough passageway for the elongated rod, such that the elongated rod can be seated in the adequate position rapidly.

In view of the aforementioned technical problems, the objective of the present disclosure provides a puncture guiding device which can adjust a first adjusting unit of a guiding sleeve and a second adjusting unit of a swing rod according to the change of a curve of the elongated rod, so that the relative position can be adjusted to enable the elongated rod to move around the pivot as a circular arc.

In view of the aforementioned technical problems, the objective of the present disclosure further provides a puncture guiding device which applies the simple members to penetrate numerous sections of spines, so that the cost of the related apparatuses can be effectively lowered. In addition, the puncture guiding device of the present disclosure can cooperate with the minimal invasive surgery to reduce the probability of complications.

In view of the aforementioned technical problems, the objective of the present disclosure further provides a surgery device assembly for minimally invasive spinal surgery, and the improved surgery device assembly resolves the complicated use of the operating problem in process of the surgery. The surgery device assembly also applies the simple members to penetrate numerous sections of spines, so that the cost of the related devices can be effectively lowered. In addition, the puncture guiding device of the present disclosure can cooperate with the minimal invasive surgery to reduce the probability of having complications.

In accordance with the aforementioned objectives, the present disclosure provides a pedicle screw which may include a bone screw body, a trough portion, and a simulation portion. The bone screw body may be a self-tapping screw thread so as to be drilled into the spine. The trough portion may be applied to accommodate an elongated rod, and may include a bottom base and a U-type trough. The bottom base may be disposed with a through hole in which the bone screw body punctures. Two sides of the U-type trough respectively may extend along two long sidewalls of the pedicle screw. The outside wall of the U-type trough may have a circular recess, and the inside wall of the U-type trough have an internal screw thread structure. The simulation portion may have a U-type simulation groove, the simulation portion may be disposed on upper margins of the two long sidewalls, and an opening of the U-type simulation groove and an opening of the U-type trough may be in the same direction. The trough portion and the simulation portion may be integral in a body.

Preferably, the internal screw tread structure may extend to two sides of the circular recess.

Preferably, the internal screw thread structure may have a square thread form and a reverse screw thread form where the internal screw thread structure is screwed.

In accordance with the aforementioned objectives, the present disclosure further provides a parallel bridge adapted to a pedicle screw which may include two sets of clamping units and a constrained pivot joint. The two sets of clamping units respectively may clamp a pedicle screw. Each clamping unit may include a fastening arm, a movable arm, and a spring. The fastening arm and the movable arm may form an aperture to hold the long sidewalls of the pedicle screw, and the spring may be disposed between the fastening arm and the movable arm. The constrained pivot joint may be connected to the fastening arm of each clamping unit, and the constrained pivot joint may limit a degree of freedom of the fastening arm to make a U-type simulation groove of each pedicle screw on the same parallel plane.

Preferably, a thickness between the fastening arm and the movable arm may be greater than 0.5 cm.

Preferably, the parallel bridge may further include a third clamping unit holding a third pedicle screw. The third clamping unit may be connected to the parallel bridge by a linkage so as to keep parallel between the U-type trough of the third pedicle screw and the U-type trough of one of the two adjacent pedicle screws.

Preferably, a gap between the aperture and the long sidewalls of the pedicle screw is provided to adjust a direction of the U-type simulation groove of the pedicle screw.

In accordance with aforementioned objectives, the present disclosure provides a puncture guiding device punctured a pedicle screw to guide an elongated rod to perform the percutaneous minimal invasive spinal fusion surgery, including: a drawbar, one end of the drawbar disposed with a handle and the other end of the drawbar having a screw thread structure, and the screw thread structure screwed to the pedicle screw to fix a position; a guiding sleeve being a hollow tube in which the drawbar is penetrated and fixed, the guiding sleeve further including a connecting portion and a pivot, and the connecting portion tabling with two sidewalls of the pedicle screw; a guiding arm including a swing rod, an arcuate bar, and a flexible wire, one end of the swing rod connected to the pivot joint and the other end of the swing rod connected to the arcuate bar, and a tunnel disposed inside the arcuate bar to accommodate the flexible wire, and a clamping portion disposed at an end of the guiding arm to grip the elongated rod; wherein a radius of curve of the elongated rod matches a relative distance between the clamping portion and the pivot so as to guide the elongated rod to move around the pivot as a circular arc.

Preferably, the guiding sleeve may further include a first adjusting unit for adjusting a relative position between the pivot and the guiding sleeve.

Preferably, the first adjusting unit may include a plurality of clamping grooves and a positioning member, and the positioning member may be abutted against any of the plurality of clamping grooves.

Preferably, the guiding sleeve may further include a clamping ring sheathed to the clamping portion to avoid the clamping portion loosening.

Preferably, the guiding arm may further include a second adjusting unit for adjusting a relative distance between the clamping portion and the pivot.

Preferably, the second adjusting unit may include a plurality of positioning grooves and an adjusting member, and the adjusting member may be abutted against any of the plurality of positioning grooves.

Preferably, the connecting portion may be a tenon structure, and the tenon structure may table with a fastening end of the pedicle screw to avoid the guiding sleeve rotating or displacing.

Preferably, the guiding arm may further include a control portion and the control portion may be abutted against the flexible wire, such that the flexible wire moves along the tunnels to release the elongated rod from the clamping portion.

Preferably, the clamping portion may be a tapered hole, a spring clip, a spring sheet set, or a rapid joint.

In accordance with the aforementioned objectives, the present disclosure further provides a surgery device assembly for minimally invasive spinal surgery. The surgery device assembly may include a plurality of pedicle screws, at least one parallel bridge, an elongated rod and a puncture guiding device. The plurality of pedicle screws are drilled into the corresponding spine respectively. Each pedicle screw may be clamped by the corresponding parallel bridge to adjust a direction of the U-type simulation groove of each pedicle screw to the same plane. The curve of the elongated rod may be adjusted according to relative height among the U-type simulation groove of each pedicle screw. The elongated rod may be disposed in the trough portion of each pedicle screw by the puncture guiding device, thereby limiting a relative distance of the corresponding spine.

According to the aforementioned description, besides the preceding advantages, the present disclosure may be able to enable store obtaining real-time price of merchandise by means of a computer system reading barcode, the consumer may also realize the real-time price of the merchandise roughly so as to become an effective media among store, merchandise and consumer.

### BRIEF DESCRIPTION OF THE DRAWINGS

FIG. 1 is a schematic diagram of a pedicle screw of the present disclosure.

FIG. 2 is the first schematic diagram of a parallel bridge of the present disclosure.

FIG. 3 is the second schematic diagram of a parallel bridge of the present disclosure.

FIG. 4 is the third schematic diagram of a parallel bridge of the present disclosure.

FIG. 5 is a schematic diagram of a puncture guiding device of the present disclosure.

FIG. 6 is a schematic diagram of a first adjusting unit and a second adjusting unit of the puncture guiding device of the present disclosure.

FIG. 7 is a schematic diagram of a clamping ring of the puncture guiding device of the present disclosure.

FIG. 8 is a schematic diagram of a surgery device assembly for minimally invasive spinal surgery.

### DETAILED DESCRIPTION OF THE PREFERRED EMBODIMENTS

Hereinafter, embodiments of the present disclosure will be described in detail with reference to the accompanying drawings so that those skilled in the art to which the present disclosure pertains can realize the present disclosure. As those skilled in the art would realize, the described embodiments may be modified in various different ways, all without departing from the spirit or scope of the present disclosure.

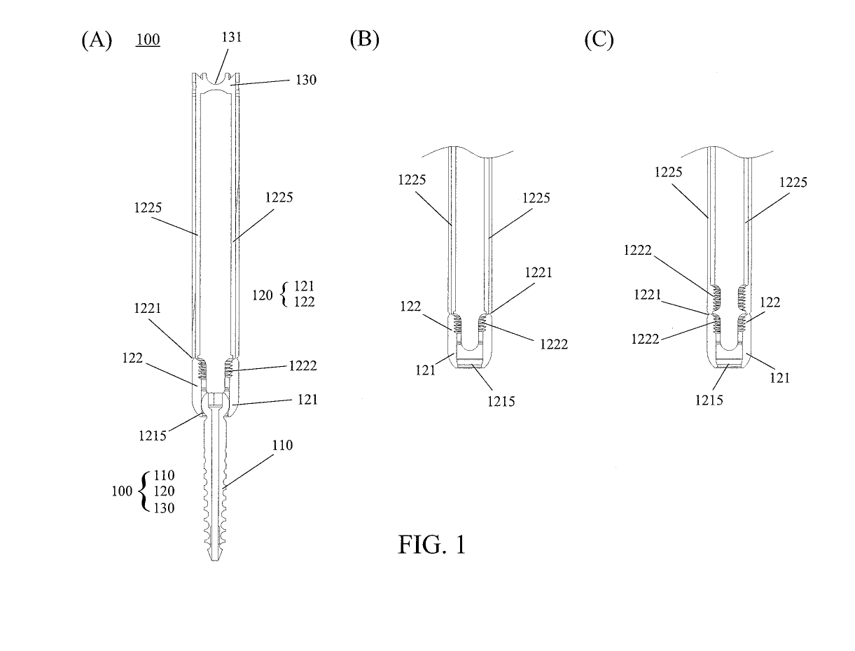

Please refer to FIG. 1, as shown in the figure, a pedicle screw 100 includes a bone screw body 110, a trough portion 120, and a simulation portion 130. The bone screw body 110, which is a self-tapping screw thread designed with a hollow guiding hole, is drilled on a target spine by a guiding needle. The trough portion 120 is applied to accommodate an elongated rod, and includes a bottom base 121 and a U-type trough 122. The bottom base 121 is disposed with a through hole 1215 in which the bone screw body 110 fastened. In practice, the bone screw body 110 and the trough portion 120 are connected to each other by a ball-and-socket structure, facilitating them to deflect with a certain angle.

Two sides of the U-type trough 122 of the trough portion 120 respectively extend upward along two long sidewalls 1225 to form a guiding passageway which enables to accommodate the other surgical instruments passing therethrough. The U-type trough 122 is further disposed with a circular recess 1221. One side of the circular recess 1221 has an internal screw thread structure 1222 adjacent to the U-type trough 122, as shown in part B of FIG. 1. Preferably, the internal screw thread structure 1222 extends to two sides of the circular recess 1221 to increase the number of thread to enhance the locking accuracy when the nut is fastened into the thread structure, and to lift the sunken vertebral body, as shown in part C of FIG. 1. In practice, the internal screw thread structure 1222 can be designed as a square thread form and having a reverse screw thread where the internal screw thread structure 1222 is screwed to enhance the coupling strength when the nut member is fastened, such that it can avoid the elongated rod loosened.

The simulation portion 130 is disposed on upper margins of the two long sidewalls 1225 and has a U-type simulation groove 131. The U-type simulation groove 131 and the U-type trough 122 have the same direction with respect to the opening, facilitating the clinical personnel to drill the pedicle screw 100 into the U-type trough 122 disposed on the patient's spine instinctively according to the opening direction of the U-type simulation groove 131. In practice, the simulation portion 130 and the trough portion 120 may be integral in a body or connected by assembling. If the simulation portion 130 and the trough portion 120 are integral in a body, it is most conventionally to mill the U-type trough 122 and then to process the U-type simulation groove 131 at the simulation portion 130.

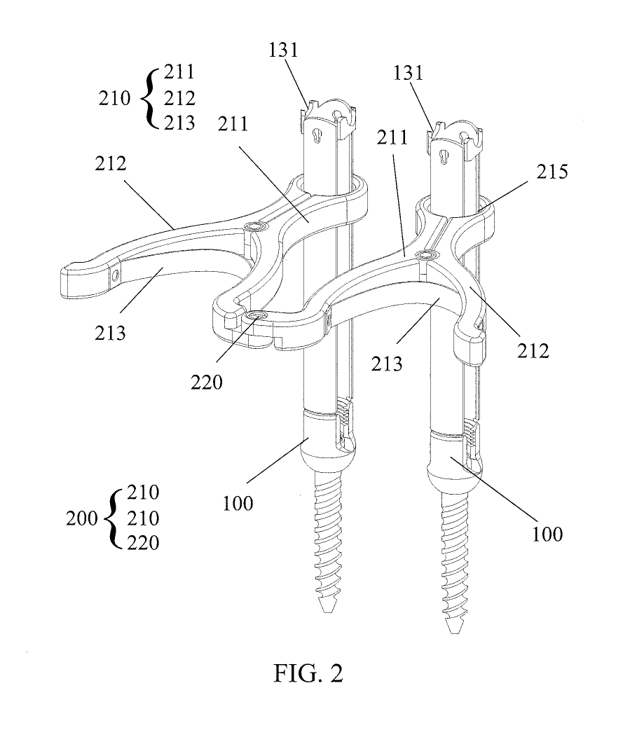

Please refer to FIG. 2 which is the first schematic diagram of a parallel bridge of the present disclosure. As shown in the figure, a parallel bridge 200 includes two sets of clamping units 210 and a constrained pivot joint 220. Each clamping unit 210 includes a fastening arm 211, a movable arm 212, and a spring 213. The fastening arm 211 and the movable arm 212 form an aperture 215 to hold the long sidewalls of the pedicle screw 100. The spring 213 is disposed between a rear end of the fastening arm 211 and a rear end of the movable arm 212, wherein a thickness between the fastening arm 211 and the movable arm 212 has to be greater than 0.5 cm so as to increase the contact surface. The constrained pivot joint 220 is connected to the fastening arm 211 of the clamping units 210. The constrained pivot joint 220 limits the movement between long sidewalls 1225 of the pedicle screw 100 which is clamped by two fastening arms 211, so as to further correct two U-type simulation grooves 131 of two pedicle screws 100 to facilitate them to be the same parallel plane. In practice, the openings are in the same direction and align to each other. However, it shall be not limited thereto, and a slight deviation without affecting the entire puncture steps is acceptable.

In practice, when the pedicle screw 100 is drilled into the spine, the flexibility of the human tissue may lead the pedicle screw 100 to produce the inclined angle, and the parallel bridge 200 is therefore applied to clamp the pedicle screw 100. The clamping unit 210, which has been adjusted parallel thereof, clamps inclined the pedicle screw 100 to maintain its parallel, and then a small clamping gap between the aperture 215 and the pedicle screw 100 is applied to facilitate the surgeon to manually adjust the direction of the U-type trough 122 of the pedicle screw 100.

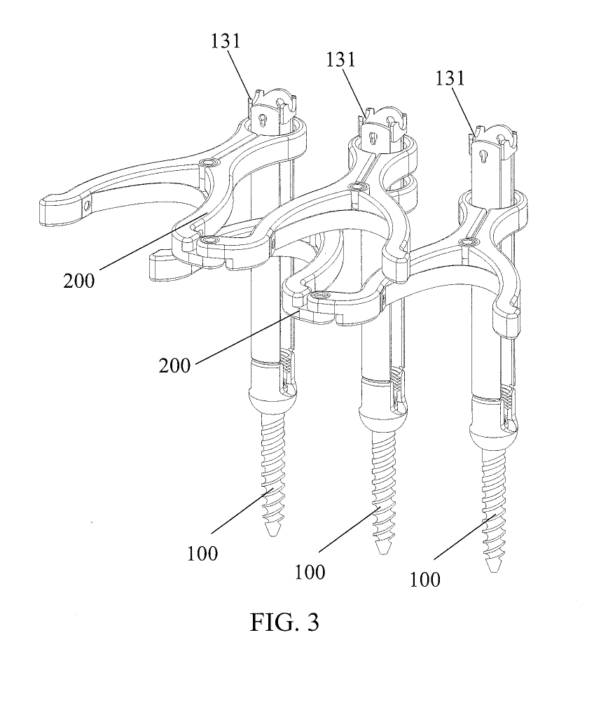

Please refer to FIG. 3 which is the second schematic diagram of a parallel bridge of the present disclosure. When numerous sections of spines have to be performed the spinal fusion surgery, a plurality of pedicle screws 100 are respectively drilled into the spines. Here, the parallel of the pedicle screw 100 plays a crucial role in terms of the success rate of the operation. When the target spines are adjacent to one another, a plurality of parallel bridges 200 are applied to clamp these target spines sequentially. By arranging the plurality of parallel bridges 200 alternating upward and downward, it can ensure the parallel of each pedicle screw 100. In practice, an amount of the parallel bridges 200 can be increased or decreased according to the actual requirements.

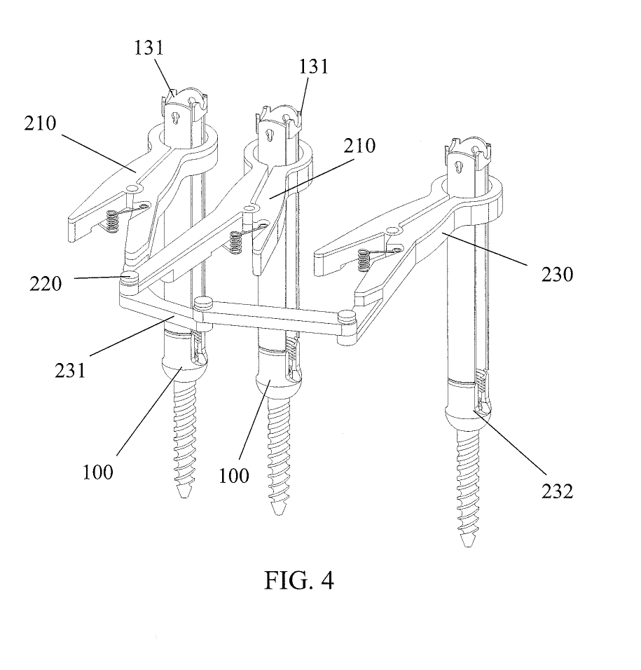

Please refer to FIG. 4 which is the third schematic diagram of a parallel bridge of the present disclosure. When two targeting spines have a farther distance, the parallel bridge 200 is applied to clamp the pedicle screw 100, and then a third clamping unit 230 is applied to clamp a third pedicle screw 232. Afterwards, a linkage 231, which is connected to the parallel bridge 200, is applied to manually adjust the relative position among each pedicle screw 100. By means of the parallel bridge 200 of the present disclosure, opening direction of the U-type simulation groove 131 of each pedicle screw 100 is capable of being adjusted to the same parallel plane to resolve the conventional technical problem concerning that the parallel interpretation has to be determined according the surgeon's clinical experience and the actual operation situation. As a result, when the elongated rod is applied to puncture, the precision and accuracy can be promoted.

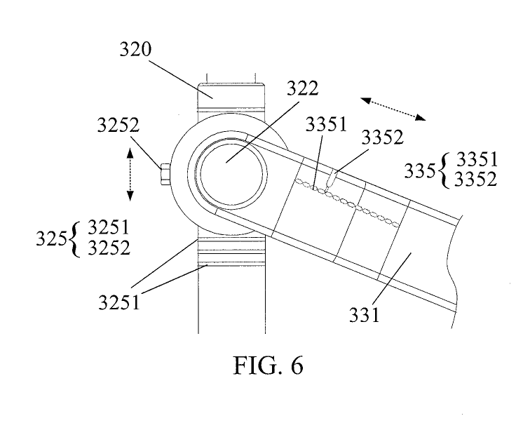

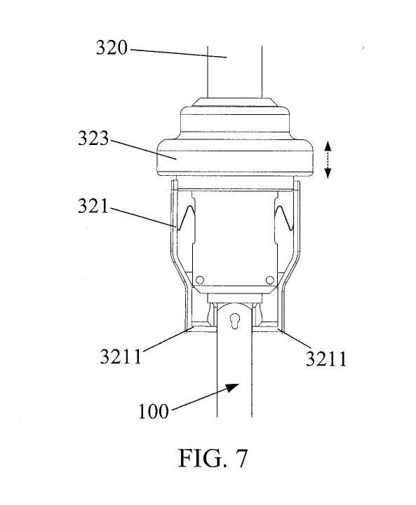

Please refer to FIG. 1 and FIG. 5 to FIG. 8 together. As shown in the figures, a puncture guiding device 300 is sheathed and fixed to a pedicle screw 100 to guide an elongated rod 400 to perform the percutaneous minimal invasive spinal fusion surgery. The puncture guiding device 300 includes a drawbar 310, a guiding sleeve 320, a guiding arm 330 and a clamping portion 340. One end of the drawbar 310 has a handle 311, and the handle 311 can be designed as T-shape or L-shape to benefit the surgeon from the operation. The other end of the drawbar 310 has a screw thread structure 312, and the screw thread structure 312 is screwed inside an internal screw thread structure 1222 of the pedicle screw 100 to fix and locate the drawbar 310.

The guiding sleeve 320 is a hollow tube and its diameter is greater than the drawbar 310, so that the drawbar 310 can be sheathed and fixed therein. The guiding sleeve 320 further includes a connecting portion 321 and a pivot 322. A tenon structure 3211 of the connecting portion 321 tables with two sidewalls of the pedicle screw 100 or a shape of a simulation portion of the pedicle screw 100 to avoid the guiding sleeve 320 producing rotation or displacement. In practice, the tenon structure 3211 of the connecting portion 321 can be designed as single or in pairs. In addition, the guiding sleeve 320 further includes a clamping ring 323. When the tenon structure 3211 is clamped to the two sidewalls of the pedicle screw 100 or the shape of the simulation portion of the pedicle screw 100, the clamping ring 323 is moved downward to limit the movement of the connecting portion 321 and to avoid the connecting portion 321 loosening, such that a relative position between the pedicle screw 100 and the guiding sleeve 320 can be fixed firmly.

To be precise, the guiding sleeve 320 further includes a first adjusting unit 325 for adjusting a relative position between the guiding sleeve 320 and the pivot 322. The first adjusting unit 325 includes a plurality of clamping grooves 3251 and positioning members 3252. The positioning members 3252 are abutted against any of the clamping grooves 3251. The clamping grooves 3251 are the structural feature on the guiding sleeve 320 and can be a circular distribution or a local arrangement. The positioning members 3252 are the members for positioning and adjusting such as socket set screws, adjusting bolts, positioning pins, and so on. When adjusting the position of the pivot 322, the positioning members 3252 are reversely screwed to facilitate the pivot 322 being adjusted to the desired height smoothly, and then the positioning members 3252 are screwed to fix the pivot 322 on the position of the guiding sleeve 320.

The guiding arm 330 includes a swing bar 331, an arcuate bar 332 and a flexible wire 333. One end of the swing bar 331 is connected to two sides of the pivot 322 by a U-type linkage, facilitating the swing bar 331 to circle the pivot 322. The arcuate bar 332 is connected to the other end of the swing bar 331, and tunnels 3321 are disposed inside the arcuate bar 332 and have the same curve as the arcuate bar 332 so as to accommodate the flexible wire 333. Furthermore, the guiding arm 330 further includes a second adjusting unit 335 to adjust a relative position between the clamping portion 340 and the pivot 322. The second adjusting unit 335 includes a plurality of positioning grooves 3351 and adjusting members 3352. The positioning grooves 3351 are disposed on the swing bar 331, and the adjusting members 3352 are abutted against any of the positioning grooves 3351. The adjusting members 3352 are the members for positioning and adjusting such as socket set screws, adjusting bolts, positioning pins, and so on.

The clamping portion 340 is disposed at the end of the guiding arm 330 to clamp the elongated rod 400. The radius of curve of the elongated rod 400 matches the relative distance between the clamping portion 340 and the pivot 322, such that the elongated rod 400 circles the pivot 322 in favor of the elongated rod 400 successfully penetrating the trough portion of the pedicle screw 100. In practice, the clamping portion 340 can be a Morse taper hole having the self-locking function, a spring clip, a spring sheet set, or a rapid joint.

To be more precise, the guiding arm 330 includes a control portion 338. The control portion 338 is disposed on the arcuate bar 332 to be connected to the flexible wire 333. When the control portion 338 actuates, the flexible wire 333 moves forward along the tunnels 321 to push the elongated rod 400 away from the clamping portion 340, such that the purpose of decoupling the elongated rod 400 is achieved.

For example, when three adjacent vertebral body are performed the spinal fusion surgery, a guide-pin is applied to show starting point of the target vertebral body sequentially, and then each pedicle screw 100 is punctured respectively. On account of the flexibility of the human tissues such as skin, muscle, and so on, each pedicle screw 100 may produce the inclined angle. Therefore, each parallel bridge 200 is applied to adjust the parallelism of respectively pedicle screws 100. Afterwards, the curve of the elongated rod 400 is gradually adjusted according to the relative height among each spine, facilitating the elongated rod 400 to match the curve of each spine. When the curve of the elongated rod 400 is adjusted, the drawbar 310 of the puncture guiding device 300 is screwed in an adequate position where the pedicle screw 100 is fastened, and then the connecting portion 312 are fastened with the sidewall of the U-type trough 122of the pedicle screw 100. The U-type trough of each pedicle screw 100 is respectively adjusted on the same parallel plane. Afterwards, the relative position between the first adjusting unit 325 and the second adjusting unit 335 is adjusted adequately to facilitate the guiding arm 330 to move around the pivot 322 as a circular arc.

Besides, the guiding arm 330 is lifting upward the pivot 322 to increase the operating space of the apparatus. The elongated rod is gripped by the clamping portion 340, and the puncture guiding device 300 is applied to tentatively penetrate the trough portion 120 of the pedicle screw 100. Next, the cone is applied to peel the adjacent muscle tissue to form a trough passageway which can accommodate the elongated rod 400. When an adequate trough passageway is prepared, the elongated rod 400 can be placed in the adequate position rapidly.

When the elongated rod 400 is guided to the trough portion of each pedicle screw 100, the surgeon operates the control portion 338 of the guiding arm 330 to push the elongated rod 400 to separate from the clamping portion 340, so that the elongated rod 400 is seated in the trough portion of each pedicle screw 100. Afterwards, the connection relationship between the puncture guiding device 300 and the pedicle screw 100 is released via a reverse operation, and the nut members of the pedicle screw 100 are sequentially fastened in the internal screw thread structure of trough portion of the pedicle screw 100 to secure the elongated rod 400 in order to stabilize the bony structure. Finally, the long sidewalls of the pedicle screw 100 are cut and removed, and then the wound is sewed to complete the entire operating procedure.

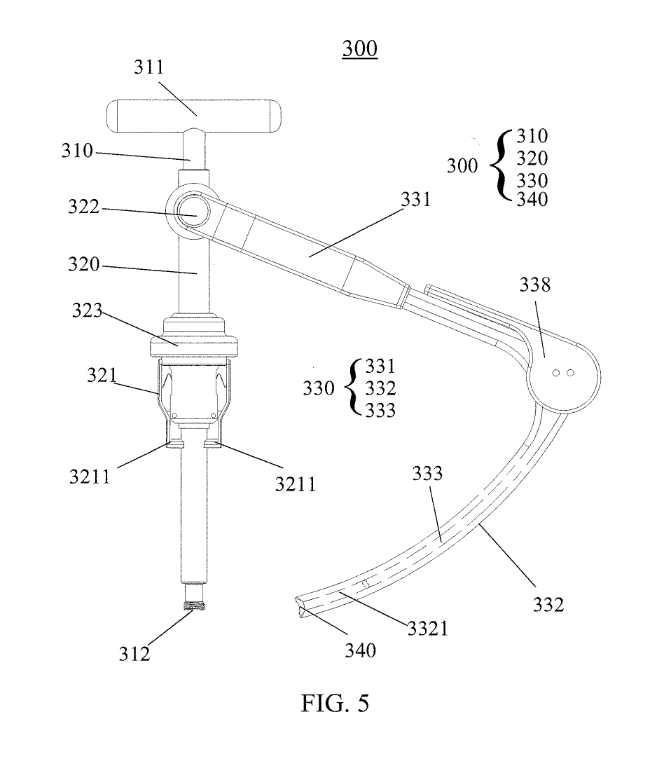

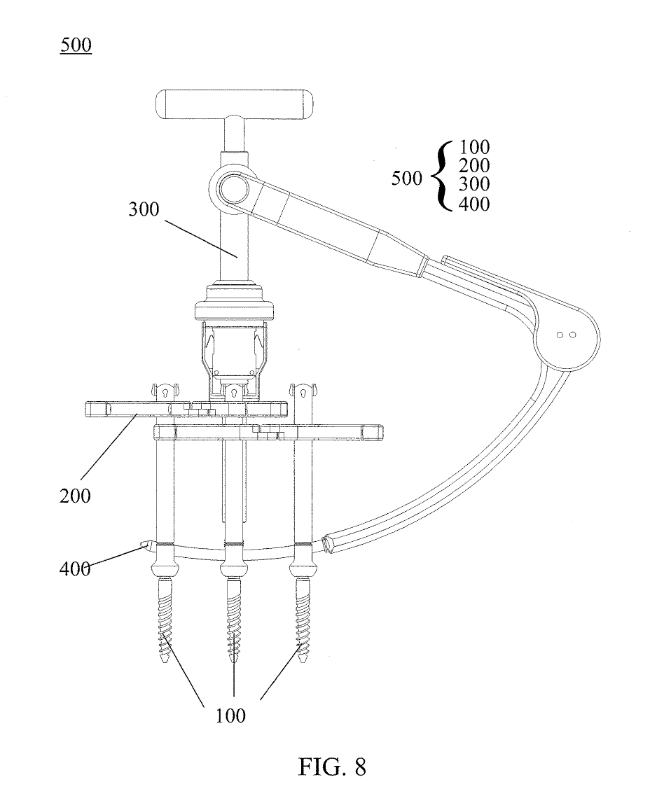

Please refer to FIG. 1, FIG. 5 and FIG. 8 together. As shown in the figures, the surgery device assembly 500 for minimally invasive spinal surgery includes a plurality of pedicle screws 100, at least one parallel bridge 200, an elongated rod 400 and a puncture guiding device 300. The present disclosure aims at improving the conventional long-tail pedicle screw. The pedicle screws 100 are made by improving the conventional long-tail pedicle screw. By means of the simulation portion, the surgeon is able to observe instinctively the relative position among the target spines so as to adjust the curve of the elongated rod. In addition, by means of the parallel bridge, a plurality of pedicle screws can be held on the same parallel plane in favor of the penetrate by the puncture guiding device 300, and setting of the elongated rod 400 so as to increase efficiency and the success rate of the surgery.

The puncture guiding device 300 of the present disclosure applies the connecting portion 321 to firmly ensure the connection relationship between the puncture guiding device 300 and the pedicle screw 100. In addition, by cooperating with the parallel bridge 200, the U-type trough 122 of each pedicle screw 100 can be maintained on the same parallel plane in favor of the follow-up puncture steps. In practice, the minor deviation of the parallel without affecting the follow-up puncture steps is acceptable. Adjusting the relative distance among each member by adjusting the first adjusting unit 325 and the second adjusting unit 335 facilitates the guiding arm 330 to move along the arc locus.

The surgery device assembly of the present disclosure is capable of assisting the surgeon to perform the percutaneous minimal invasive spinal fusion surgery more precisely without the other complicated operating procedures, so that the entire operating time is shortened, the probability of having complications is decreased, and the cost of the related apparatus is reduced effectively.

While the means of specific embodiments in present disclosure has been described by reference drawings, numerous modifications and variations could be made thereto by those skilled in the art without departing from the scope and spirit of the disclosure set forth in the claims. The modifications and variations should in a range limited by the specification of the present disclosure.

What is claimed is:

1. A pedicle screw adapted to accommodate an elongated rod, comprising:
a bone screw body;
a trough portion for accommodating the elongated rod comprising a bottom base and a U-type trough, the bottom base disposed with a through hole in which the bone screw body punctures, two sides of the U-type trough respectively extending along two long sidewalls, the outside wall of the U-type trough having a circular recess, and the inside wall of the U-type trough having an internal screw thread structure; and
a simulation portion having a U-type simulation groove, and the simulation portion disposed on upper margins of the two long sidewalls, wherein an opening of the U-type simulation groove and an opening of the U-type trough are in the same direction;
wherein, the trough portion and the a simulation portion are integrated in a body.

2. The pedicle screw of claim 1, wherein the internal screw thread structure extends to two sides of the circular recess.

3. The pedicle screw of claim 1, wherein the internal screw thread structure has a square thread form and a reverse screw thread where the internal screw thread structure is screwed.

4. A parallel bridge for keeping two adjacent pedicle screws parallel to each other, comprising:
two sets of clamping units respectively clamping a pedicle screw, each clamping unit comprising a fastening arm, a movable arm, and a spring, the fastening arm and the movable arm forming an aperture to hold the long sidewalls of the pedicle screw, and the spring disposed between the fastening arm and the movable aim, and
a constrained pivot joint connected to the fastening arm of each clamping unit, the constrained pivot joint limited a degree of freedom of the fastening arm to make a U-type simulation groove of each pedicle screw on the same parallel plane.

5. The parallel bridge of claim 4, wherein a thickness between the fastening arm and the movable arm is greater than 0.5 cm.

6. The parallel bridge of claim 4, further comprising a third clamping unit holding a third pedicle screw, and the third clamping unit connected to the parallel bridge by a linkage so as to keep parallel between the U-type trough of the third pedicle screw and the U-type trough of one of the two adjacent pedicle screws.

7. The parallel bridge of claim 4, wherein a gap between the clamping portion and the long sidewalls of the pedicle screw is provided to adjust a direction of a U-type simulation groove of the pedicle screw.

8. A puncture guiding device punctured through a pedicle screw to guide an elongated rod to perform the percutaneous minimal invasive spinal fusion surgery, comprising:
a drawbar, one end of the drawbar disposed with a handle and the other end of the drawbar having a screw thread structure, and the screw thread structure screwed into a U-type trough of the pedicle screw to fix to a position;
a guiding sleeve being a hollow tube in which the drawbar is penetrated and fixed, the guiding sleeve further comprising a connecting portion and a pivot, and the connecting portion fastened to the sidewalls of the U-type trough of the pedicle screw;
a guiding arm comprising a swing rod, an arcuate bar, and a flexible wire, one end of the swing rod connected to the pivot and the other end of the swing rod connected to the arcuate bar, and a tunnel disposed inside the arcuate bar to accommodate the flexible wire, and
a clamping portion disposed at an end of the guiding arm to grip the elongated rod;
wherein a radius of curve of the elongated rod matches a relative distance between the clamping portion and the pivot so as to guide the elongated rod to move around the pivot as a circular arc.

9. The puncture guiding device of claim 8, wherein the guiding sleeve further comprises a first adjusting unit for adjusting a relative position between the pivot and the guiding sleeve.

10. The puncture guiding device of claim 9, wherein the first adjusting unit comprises a plurality of clamping grooves and a positioning member, and the positioning member is abutted against any of the plurality of clamping grooves.

11. The puncture guiding device of claim 8, wherein the guiding sleeve further comprises a clamping ring sheathed to the connecting portion to avoid the connecting portion loosening.

12. The puncture guiding device of claim 8, wherein the guiding arm further comprises a second adjusting unit for adjusting a relative distance between the clamping portion and the pivot.

13. The puncture guiding device of claim 12, wherein the second adjusting unit comprises a plurality of positioning grooves and an adjusting member, and the adjusting member is abutted against any of the plurality of positioning grooves.

14. The puncture guiding device of claim 8, wherein the connecting portion is a tenon structure, and the tenon structure is tabling with a simulation portion of the pedicle screw to avoid the guiding sleeve rotating or displacing.

15. The puncture guiding device of claim 8, wherein the guiding arm further comprises a control portion and the control portion is abutted against the flexible wire, such that the flexible wire moves along the tunnels to release the elongated rod from the clamping portion.

16. The puncture guiding device of claim 8, wherein the clamping portion is a tapered hole, a spring clip, a spring sheet set, or a rapid joint.

17. A surgery device assembly for minimally invasive spinal surgery, comprising:
a plurality of pedicle screws and each pedicle screw drilled into a corresponding spine; wherein each pedicle screw comprises: a bone screw body, a trough portion for accommodating an elongated rod comprising a bottom base and a U-type trough, the bottom base disposed with a through hole in which the bone screw body punctures, two sides of the U-type trough respectively extending along two long sidewalls, the outside wall of the U-type trough having a circular recess, and the inside wall of the U-type trough having an internal screw thread structure; and a simulation portion having a U-type simulation groove, and the simulation portion disposed on upper margins of the two long sidewalls, wherein an opening of the U-type simulation groove and an opening of the U-type trough are in the same direction;
at least one parallel bridge clamping the corresponding pedicle screws to adjust the U-type simulation groove of each pedicle screw on the same parallel plane, wherein the at least one parallel bridge comprises two sets of clamping units respectively clamping the corresponding pedicle screws, each clamping unit comprising a fastening arm, a movable aim, and a spring, the fastening arm and the movable arm forming an aperture to hold the long sidewalls of the pedicle screw, the spring disposed between the fastening arm and the movable arm; a constrained pivot joint connected to the fastening arm of each clamping unit; and the constrained pivot joint limiting a degree of freedom of the fastening arm to make a U-type simulation groove of each pedicle screw on the same parallel plane;
a puncture guiding device which punctures through the pedicle screw to guide the elongated rod to perform the percutaneous minimal invasive spinal fusion surgery, the puncture guiding device comprising a drawbar, a guiding sleeve, a guiding arm and a clamping portion; wherein one end of the drawbar disposes with a handle and the other end of the drawbar has a screw thread structure, and the screw thread structure screw to into the U-type trough of the pedicle screw to fix to a position; the guiding sleeve is a hollow tube in which the drawbar is penetrated and fixed, the guiding sleeve further comprises a connecting portion and a pivot, and the connecting portion fastened to the sidewalls of the U-type trough of the pedicle screw; the guiding arm comprises a swing rod, an arcuate bar, and a flexible wire, one end of the swing rod connects to the pivot and the other end of the swing rod connects to the arcuate bar, and a tunnel disposes inside the arcuate bar to accommodate the flexible wire, and the clamping portion disposes at an end of the guiding arm to grip the elongated rod; wherein a radius of curve of the elongated rod matches a relative distance between the clamping portion and the pivot so as to guide the elongated rod to circle; and
the elongated rod;
wherein, a curve of the elongated rod is adjusted according to a connection curve of the U-type trough groove of each pedicle screw, and the elongated rod seated in the trough portion of each pedicle screw by the puncture guiding device, the elongated rod is secured to nuts in order to stabilize a bony structure.
